(19) United States
(12) Patent Application Publication
(10) Pub. No.: US 2014/0350643 A1
(43) Pub. Date: Nov. 27, 2014

(54) PHOTOTHERAPY APPARATUS FOR SKIN TREATMENT

(71) Applicant: Apira Science, Inc., Irvine, CA (US)

(72) Inventors: Morgan Pepitone, Irvine, CA (US); Jeffrey Braile, Irvine, CA (US); Nicholas Brox, Irvine, CA (US); Gavin Tucker, Irvine, CA (US)

(73) Assignee: Apira Science, Inc., Irvine, CA (US)

(21) Appl. No.: 14/286,061

(22) Filed: May 23, 2014

Related U.S. Application Data
(60) Provisional application No. 61/826,687, filed on May 23, 2013.

(51) Int. Cl. A61N 5/06

(52) U.S. Cl. CPC A61N 5/0616; A61N 5/0617; A61N 2005/0647; A61N 2005/0652; A61N 2005/0659; A61N 2005/067
USPC 607/89; 607/91

(57) ABSTRACT

A wearable hands-free apparatus for providing phototherapy treatment to a number of hair, scalp and skin related conditions includes a supporting member for pivotally supporting a light emitting plate in spaced, opposing relation to the user's head. The light emitting plate is fitted with an array of light generating sources, such as light emitting diodes (LEDs), laser diodes, or infrared lights, that emit light within a particular wavelength range correlating with the treatment of one or more specific hair, scalp and/or skin-related conditions. The light emitting plate is specifically designed to conform to the shape of the human face or scalp for providing complete, uniform and consistent light coverage to the respective areas.

This patent application is based on provisional patent application Ser. No. 61/826,687 filed on May 23, 2013.

### BACKGROUND OF THE INVENTION

1. Field of the Invention

This invention relates to light therapy for the treatment of skin, scalp and hair and, more particularly, to a hands-free phototherapy apparatus for providing therapeutic aid by way of evenly distributed light of various beneficial wavelengths that is directed onto particular treatment areas of a user's facial skin, scalp and/or hair.

2. Discussion of the Related Art

People are frequently confronted with hair loss as well as a variety of different scalp and skin-related conditions, such as acne, sun spots, and wrinkling of the skin, psoriasis and non-melanoma skin cancer. In response, an assortment of treatment products, each typically targeting one specific hair, scalp or skin-related condition, have been developed over the past 75 years and made available to the public. Many of these products are in the form of a topical solution that requires an arduous application process. Where the condition is hair loss, a surgical process has been made available, wherein hair plugs are surgically transplanted in place of the missing hair. However, this surgical process for treating hair loss is extremely expensive and consequently, is not available to an average consumer.

More recently, the use of phototherapy to treat hair loss, as well as various skin and scalp disorders, has become increasingly popular. Phototherapy consists of exposure to specific wavelengths of light using lasers, light emitting diodes (LED's) (both individual and arrays), IPL's (Intense Pulsed Light) and other light sources, for a prescribed amount of time to both treat disease and affect cosmetic enhancements to the hair, scalp and skin. The use of phototherapy in medical science and aesthetics is rapidly evolving as more and more wavelengths of light are being identified to target various sections of cells in order to stimulate cellular proficiency and enhance the body's ability to heal and rejuvenate itself Phototherapy is currently used to treat acne, wrinkles, sun and age spots, rosacia, eczema, hair loss and wound healing through wavelengths indicated by various colors (i.e., wavelengths) of the light spectrum. By utilizing various wavelengths, colors relatively close on the spectrum can cause different effects when applied to various parts on the body.

For example, red light at a wavelength of 670 nanometers has been clinically shown to prevent hair loss and re-grow new hair, as well as to cause increased melanin production and protein synthesis. Red and infrared lights have also been used to increase the production of collagen and to reduce redness, dilated capillaries and damage to the skin, as well as reduction of wrinkles and fine lines. Blue light has been clinically shown to reduce acne and, when combined with red light, eliminates acne and reduces the scarring often associated with acne treatment. Yellow and Amber lights have been clinically shown to reduce fine lines and wrinkles, rosacia, and can help to repair sun damaged skin. Green light has been shown to reduce and eliminate sun and age spots, lighten freckles and also help promote more luminous skin condition and overall radiance of the skin. As set forth above, many of these light sources have multiple benefits, cross over each other in treating certain ailments and work to promote a variety of benefits to the hair and skin. These light sources are often used in combinations to provide increase efficacy and various degrees of stimulation.

Science throughout the years has determined the effects of various wavelengths of light, but absorption is the key to cellular change. Light therapy emits photons which are absorbed by the skins photoreceptors. Hair and skin cells respond well to phototherapy involving low level light due to the fact that cells reside just underneath the skin surface, making these low levels of energy able to reach the receptor sites and induce photochemistry.

There are a number of phototherapy devices currently available for home use to treat both skin and hair. The majority of these are hand held devices, varying in both size and number of light sources (i.e., laser diodes, LED's, or infrared diodes). These devices are manually moved around the hair or face by the user and require a constant movement in order to expose the entire surface area to the light sources. This results in an uneven treatment protocol, as the average user is unlikely to be able to cover the entire surface area through manual movements and will leave certain areas untreated. Further, due to the need for a manageable size (must fit in the hand), these devices are often underpowered.

Several phototherapy devices have been developed that are adapted to be portably worn by a user in a hands-free mode of operation. For example, U.S. Pat. App. Pub. No. 2009/0012586 A1 to Kepecs discloses a system that houses LEDs within a head unit that resembles a baseball helmet. The Kepecs device is used for reducing hair loss, as well as the therapeutic healing of a variety of skin disorders. One particular shortcoming of the Kepecs device is the onerous task of snapping or screwing in different LEDs to alter the desired wavelength.

U.S. Pat. App. Pub. No. 2006/0030908 to Powell et al. discloses a skin treatment phototherapy device that may comprise a clamshell structure, pen shape, facial mask, or desk lamp design, and which includes multi-colored LEDs. The Powell device attempts to treat a variety of skin conditions on the face and other skin regions below the user's head. Depending on the skin condition to be treated, the corresponding wavelengths, intensity levels, and time interval for the skin treatment can be varied by a control system. However, this device is neither designed nor intended to treat hair loss. Moreover, this device lacks a suitable structure and design for directing an evenly distributed light pattern upon a user's entire scalp area.

A further example of a phototherapy device that is adapted to be worn on a user's head is disclosed in PCT International Patent Application No. JP2002/009778 to Shimizu. Shimizu discloses a phototherapy device for home use that has a head band fitted with multiple LED's. The head band is structured to span over the top of a user's head, covering only a portion of the scalp. This device also provides headphones attached to the head band. The head band of the Shimizu device is moveable between two or more positions relative to the user's scalp. In order to attain total scalp coverage that is needed for effective phototherapy treatment of hair loss, the head band of the Shimizu device must be moved to the several positions. This is due to the limited size and shape of the Shimizu head band, as well as variations in the shape of the human scalp. Accordingly, the Shimizu phototherapy device requires a minimum of two movements of the head band to cover the entire scalp, with a treatment performed at each position, thereby extending the overall time of an effective phototherapy treatment session that is needed for full scalp coverage.

Presently, there are clinical or salon based laser phototherapy devices (commercial devices) that are stationary and require a user to sit beneath them at a fixed location while undergoing treatment. These stationary commercial phototherapy systems are similar in nature to stationary hair dryers that are used at women's hair salons. More specifically, clinical or salon based laser therapy devices for hair growth include a hood that is positioned over a chair. These clinical or salon based stationary phototherapy systems are the only phototherapy systems known to provide simultaneous total scalp coverage without having to move or adjust the position of the head unit (i.e., hood) relative to the user's scalp. Laser hair therapy sessions for full scalp coverage treatments, using these clinical or salon based phototherapy laser systems, are typically in the range of about 20-30 minutes long. Thus, the Shimizu portable home phototherapy device, requiring a minimum of two movements of the headband, would extend the session to between 40 and 60 minutes for full scalp coverage. This extended phototherapy session time frame is beyond the norm for home use light-based therapies which should require no more than 25 minutes.

The present invention provides the home use equivalent of the clinical stationary laser phototherapy systems in a convenient and easy to use device that provides for full facial skin or scalp coverage. Moreover, the present invention provides the added benefit of ensuring a uniformly consistent distance from each of the light emitting sources to the face or scalp. This improves on the clinical or stationary laser phototherapy systems in which the distance between each of the light emitting sources and the user's skin or scalp may vary from one person to the next due to the fact that they must adjust the hood or panel dependant on the height of the person being treated and/or the chair height.

### SUMMARY OF THE INVENTION

The present invention is directed to a wearable hands-free apparatus that provides phototherapy treatment to the facial skin tissue and scalp of a user. In one embodiment, the phototherapy apparatus includes a support member that wraps over the user's shoulders and around the user's neck to support a unique contoured plate that houses an array of light generating sources. The plate is structured and configured to provide complete and evenly distributed light to the entire scalp or face area being treated. In another embodiment, the phototherapy apparatus is securable to the user's head and includes a head cup sized and configured for engaged receipt of a portion of the user's forehead and an adjustable strap that wraps around the backside of the user's head.

For this application, the phrase “light generating sources” includes, but is not limited to, light emitting diodes (LEDs), laser diodes, infrared, and intense pulse lights (IPLs). The photo-biostimulation process achieved by use of the phototherapy apparatus of this invention produces an increase in ATP and keratin production, enhancement in blood flow and circulation, as well as an increase in collagen production. As previously noted, phototherapy can be used to treat hair loss, and a number of skin and scalp conditions, such as acne, sunspots, wrinkle reduction, skin tightening, psoriasis, eczema and collagen production.

Each form of treatment requires light emitted within a particular wavelength range in order to be sufficiently absorbed into the skin tissue, to thereby treat a user's particular skin, scalp or hair-related condition. The plate houses an array of light generating sources that are capable of emitting light within a range of output wavelengths in order to provide one or more penetration depths and photo-biostimulation effects. In a further embodiment of the invention, the plate may contain an array of mixed light generating sources, wherein certain light generating sources emit light within one wavelength range, while other light generating sources emit light within different wavelength ranges, thereby targeting different areas of the cell.

The plate is specifically designed to generally conform to the shape of the human face and scalp in order to provide complete light coverage to the face or scalp so that all areas that are most commonly affected by facial skin disorders and hair loss in both men and women can be simultaneously treated. The plate may be fixed as an integral part of the apparatus or, alternatively, may be interchangeably attached by way of a releasable securing mechanism. Various embodiments of the releasable securing mechanism utilizing different methods of interchangeable attachment are contemplated.

According to the preferred embodiment of the present invention, the bottom or inner side of the plate, that is disposed in opposing relation to the user's face or scalp, is designed to generally follow the shape and curvature of the average adult face and scalp.

The light generating sources (e.g., diodes) may be adapted to pulse according to a proprietary algorithm that is programmed in the memory of a control device. The algorithm may provide for pulsed light in specific pre-determined patterns and timing sequences in accordance with a particular skin related treatment.

### BRIEF DESCRIPTION OF THE DRAWINGS

For a fuller understanding of the nature of the present invention, reference should be made to the following detailed description taken in conjunction with the accompanying drawings in which:

FIG. 1 is a side elevational view showing the phototherapy apparatus in accordance with one embodiment of the present invention, and wherein the plate is sized, structured and configured to cover the face of a user, and wherein the phototherapy apparatus includes an array of light generating sources on an inner side of the plate for producing a light pattern that can be directed onto the user's face, and further wherein the plate is pivotally supported by a support member that wraps over the user's shoulders and/or around the user's neck;

FIG. 2 is a side elevational view showing the phototherapy apparatus in accordance with one embodiment of the present invention, and illustrating pivotal movement of the plate away from the user's face;

FIG. 3 is a side elevational view showing the phototherapy apparatus in accordance with an alternative embodiment of the present invention, and wherein the plate is sized, structured and configured to cover the scalp of a user;

FIG. 4 is a side elevational view showing the phototherapy apparatus in accordance with an alternative embodiment of the present invention, and wherein a head cup is attached to an adjustable strap support member;

FIG. 5 is side elevational view showing the phototherapy apparatus in accordance with an alternative embodiment of the present invention;

FIG. 6 is a prospective view showing the phototherapy apparatus in accordance with an alternative embodiment of the present invention, and illustrating pivotal movement of the plate away from the user's face;

FIG. 7 is a perspective view of an example of an array arrangement of light generating sources on the inner facing side of the plate;

FIG. 8 is a perspective view illustrating goggles for protecting the user's eyes;

FIG. 9 is a perspective view showing the phototherapy apparatus (without a face plate) in accordance with an alternative embodiment of the present invention on a user's head, and illustrating goggles covering the user's eyes;

FIG. 10 is a top plan view illustrating a controller for operating the phototherapy apparatus of the present invention; and

FIG. 11 is a perspective view showing the phototherapy apparatus in accordance with an alternative embodiment of the present invention, and illustrating a dial member on the adjustable strap support member.

Like reference numerals refer to like referenced parts throughout the several views of the drawings.

### DETAILED DESCRIPTION OF THE PREFERRED EMBODIMENTS

Referring to the several views of the drawings, the wearable hands-free apparatus that provides phototherapy treatment to the scalp, skin tissue, and layers of a user's dermis is shown according to several embodiments of the invention and is generally indicated as 10.

In each of the embodiments of the invention, the phototherapy apparatus 10 includes a supporting member 12 (e.g., a detachable strap) that is securable to a user for supporting a light emitting plate 14 about the user's head. The supporting member 12 supports a light emitting plate 14 that houses an array of light generating sources 102, such as light emitting diodes (LEDs), lasers, infrared lights, or other suitable light sources that are adapted to emit light within a particular wavelength range correlating with the treatment of one or more specific hair loss, scalp and/or skin-related conditions. The plate 14 is specifically designed to provide complete light coverage to the areas the human face and/or scalp.

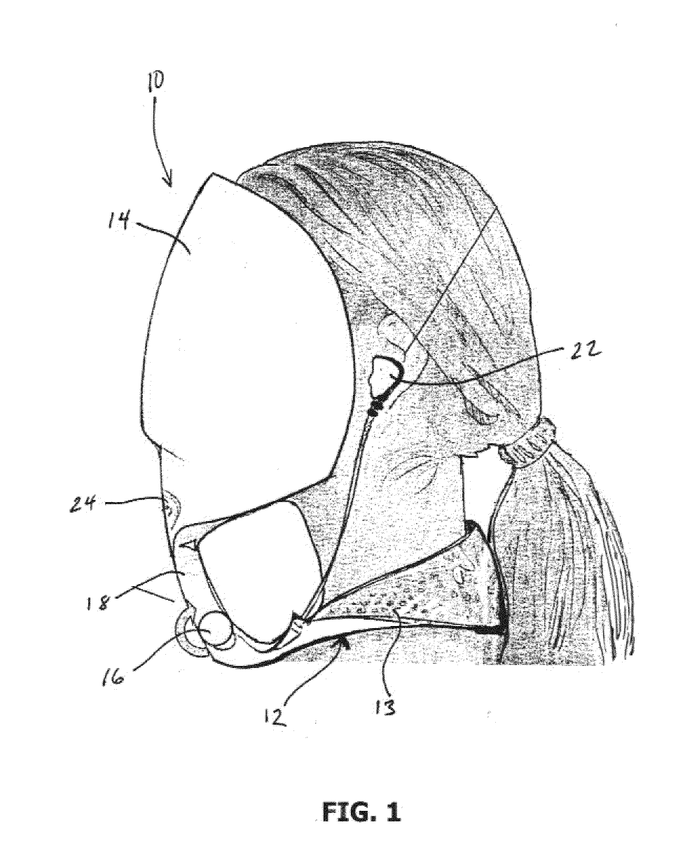

Referring to FIGS. 1-3, a first embodiment of the supporting member 12 includes detachable straps 13 that wrap over the user's shoulders and around the user's neck and are attached together (i.e. snap fastener, hook and loop fastener, buckle fastener, etc.) to support the phototherapy apparatus 10 to the user. The detachable straps 13 are made from an inner rigid material, such as plastic, and an outer flexible material, such as neoprene, which further includes perforations to allow for maximum comfort and breathability. The detachable straps 13 are securable to one another by conventional fastening means, such as a hook and loop fastener. The inner rigid material generally conforms to the portion of the user's shoulders and neck about which the supporting member 12 is in contact during use. Other embodiments of a supporting member 12 that wrap over the user's shoulders and/or neck have been considered, such as a neck strap and shoulder hooks sized and configured to support the phototherapy apparatus 10 on a user. In addition, a back support member (not pictured) may be used for added support and comfort. The back support member is sized and configured to generally conform to the backside of a user's back, neck and/or head and may be integrally formed as part of the supporting member 12 or, alternatively, detachable from the supporting member 12.

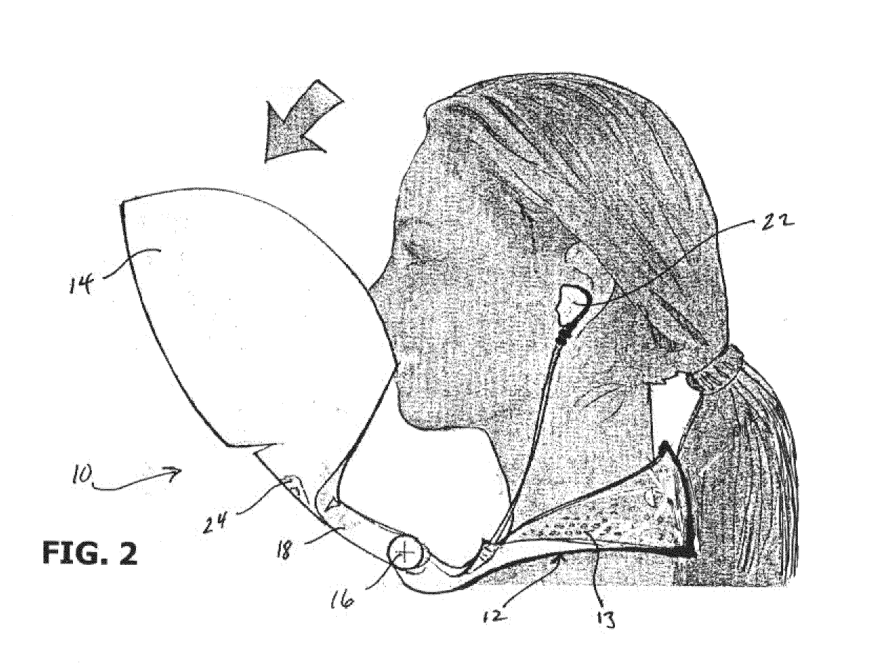

Referring to FIGS. 1 and 2, a first embodiment of the light emitting plate 14 is shown wherein the plate 14 is supported by a pivoting member 16 on the front side of supporting member 12 in connection with elongate member 18, which allows the plate 14 to be pivotally moved towards and away from the user's face, as shown in FIG. 2.

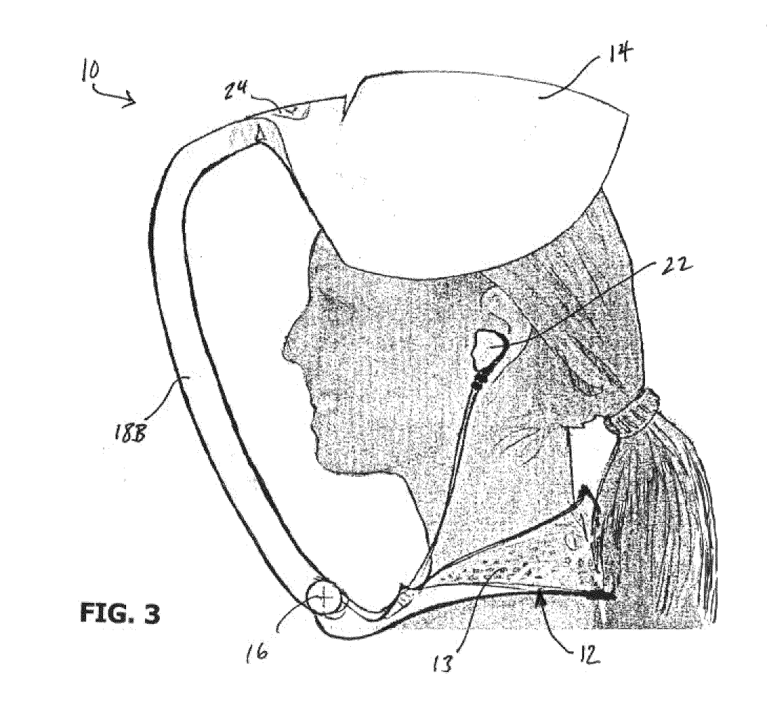

Referring to FIG. 3, an alternative embodiment of the phototherapy apparatus 10 is shown, wherein the plate 14 is pivotally held above the user's head by an extended elongate member 18B such that the light generating sources 102 are positioned to provide complete light coverage to the user's scalp.

As shown throughout the Figures, one or more audio headphones 22 may be used to listen to music or other audio programs while the user is receiving light therapy. The audio headphones 22 may be integrally attached to the phototherapy apparatus 10 or, alternatively, the phototherapy apparatus 10 may include connection ports for receiving the user's personal audio headphones 22. A PLAY/PAUSE button 24 on the outer facing side of the plate 14 is used to control the audio program.

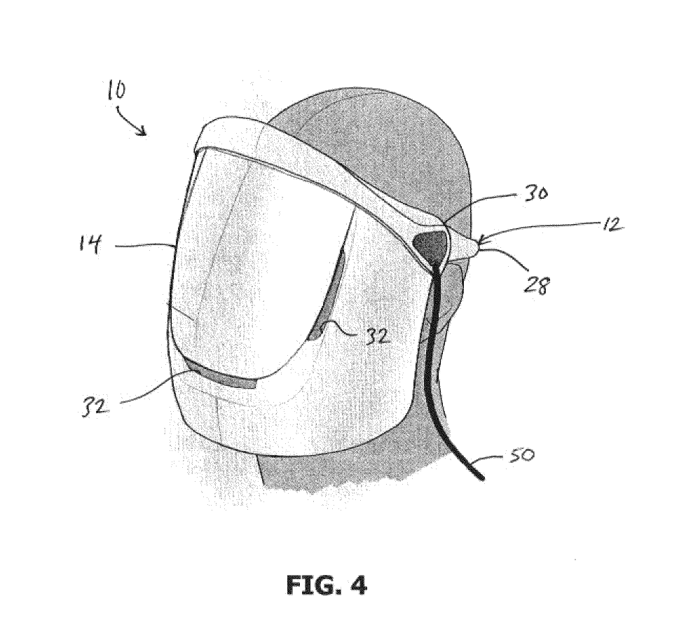

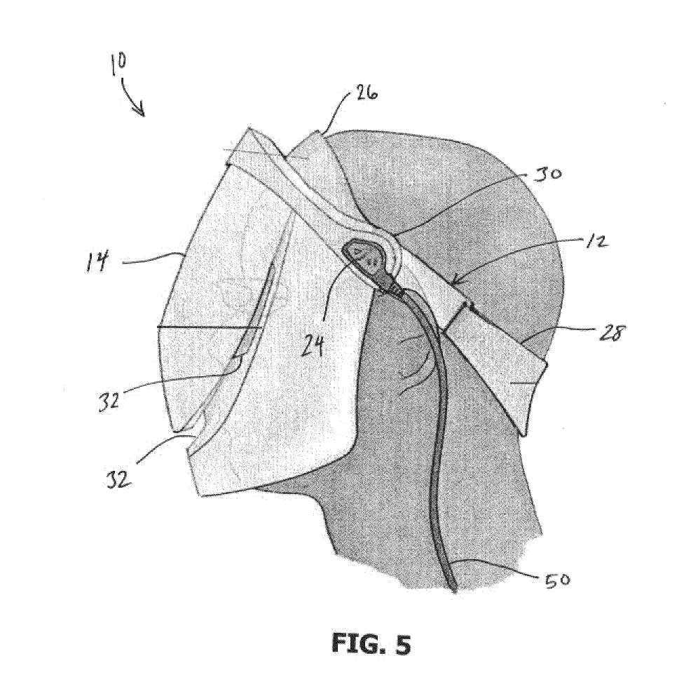

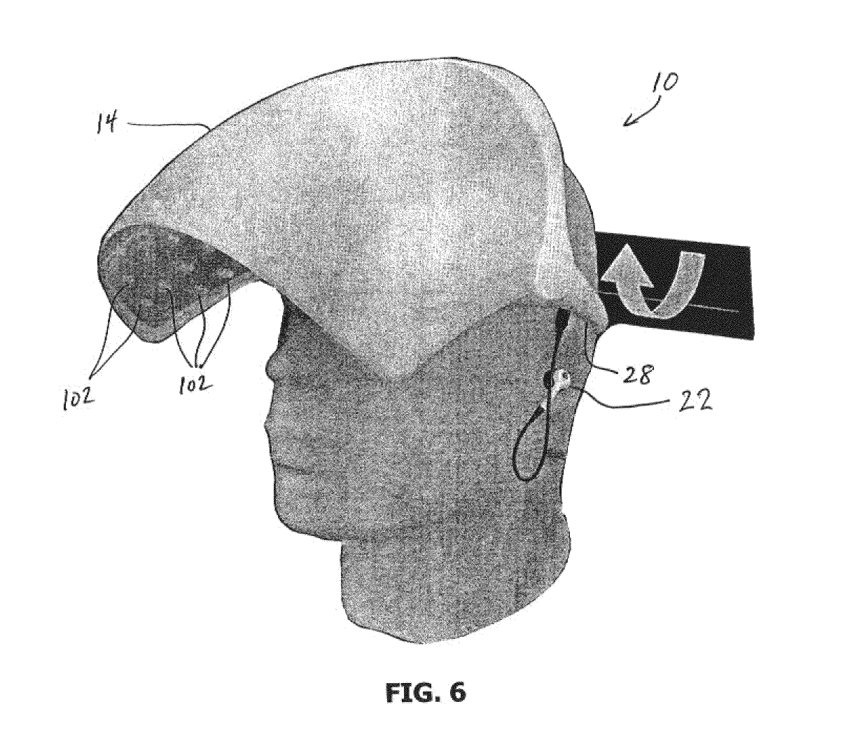

Referring to FIGS. 4-6, another embodiment of the phototherapy apparatus is shown wherein the supporting member 12 includes a head cup 26 and an integrally attached, adjustable strap 28. The head cup 26 is sized and configured (e.g., curved) for engaged receipt of a portion of the user's forehead and the adjustable strap 28 is sized to wrap around the backside of the user's head for supporting the phototherapy apparatus thereon. In a preferred embodiment of the head cup 26 and adjustable strap 28, the inner facing surface is cushioned for user comfort. The light emitting plate 14 is attached to the supporting member 12 by pivoting member 30, which allows the plate 14 to be pivotally moved towards and away from the user's face, as shown in FIG. 6. Referring to FIGS. 4 and 5, one or more vent openings 32 extending between the inner and outer facing sides of the plate 14 are provided for comfort of the user. The vent openings 32 may be positioned near the mouth and nose of the user in order to facilitate air circulation as the user breathes. Vent openings 32 near the peripheral edge of the plate 14 further facilitate the circulation of air between the inner facing side of the plate 14 and the user's face or scalp for reducing the temperature of the ambient air surrounding the user's face or scalp during operation of the phototherapy apparatus 10.

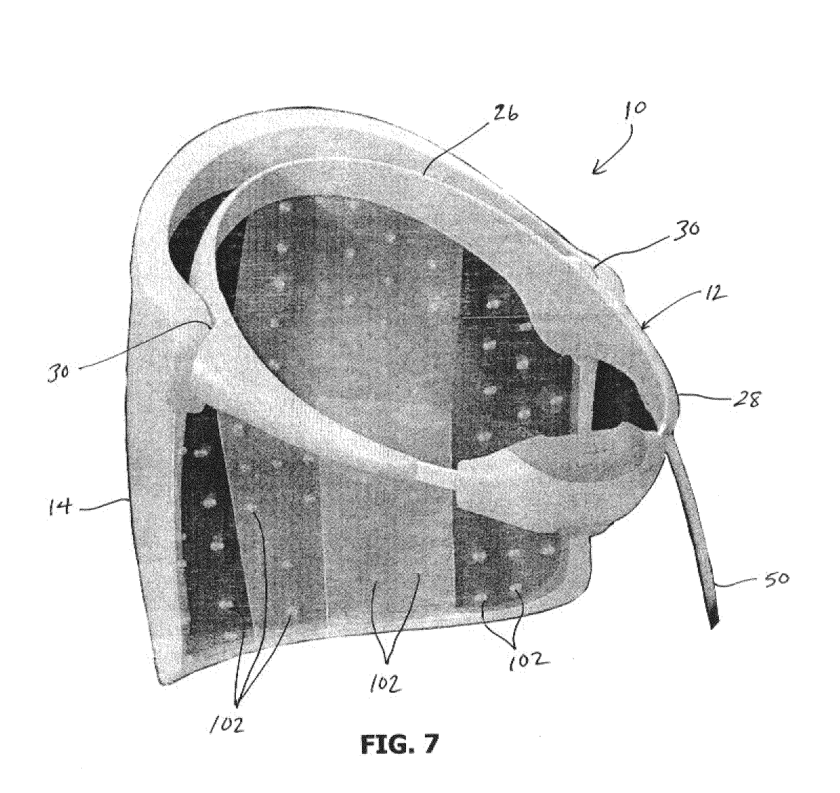

Referring to FIG. 7, the inner facing side of the light emitting plate 14 includes a plurality of light generating sources 102, such as light emitting diodes (LEDs), laser diodes, or infrared lights, and intense pulse lights. The light generating sources 102 may be arranged in varying patterns. Example arrangements of the light generating sources 102 range from a 10×30 array and a 2×150 array. In a preferred embodiment of the phototherapy apparatus 10, the electronic circuitry that controls the light emission and audio signal emission of the phototherapy apparatus 10 is internally housed between.

In each of the embodiments shown, depending on the type of condition being treated, light emitted at a particular output wavelength range is required to sufficiently penetrate the skin tissue. For example, in treating inflammation, lesions, or canker sores, a range (628 nm-694 nm) of red wavelengths is preferable; in treating rosacea or wrinkling of the skin, a range (568 nm-590 nm) of yellow wavelengths is preferable; in treating acne, a range (405 nm-476 nm) of blue wavelengths is preferable; in treating age spots, sun damage, or hyperpigmentation, a range (514 nm-543 nm) of green wavelengths is preferable; and in stimulating the skin to produce collagen and elastin, a range (700-1090 nm) of infrared wavelengths is preferable.

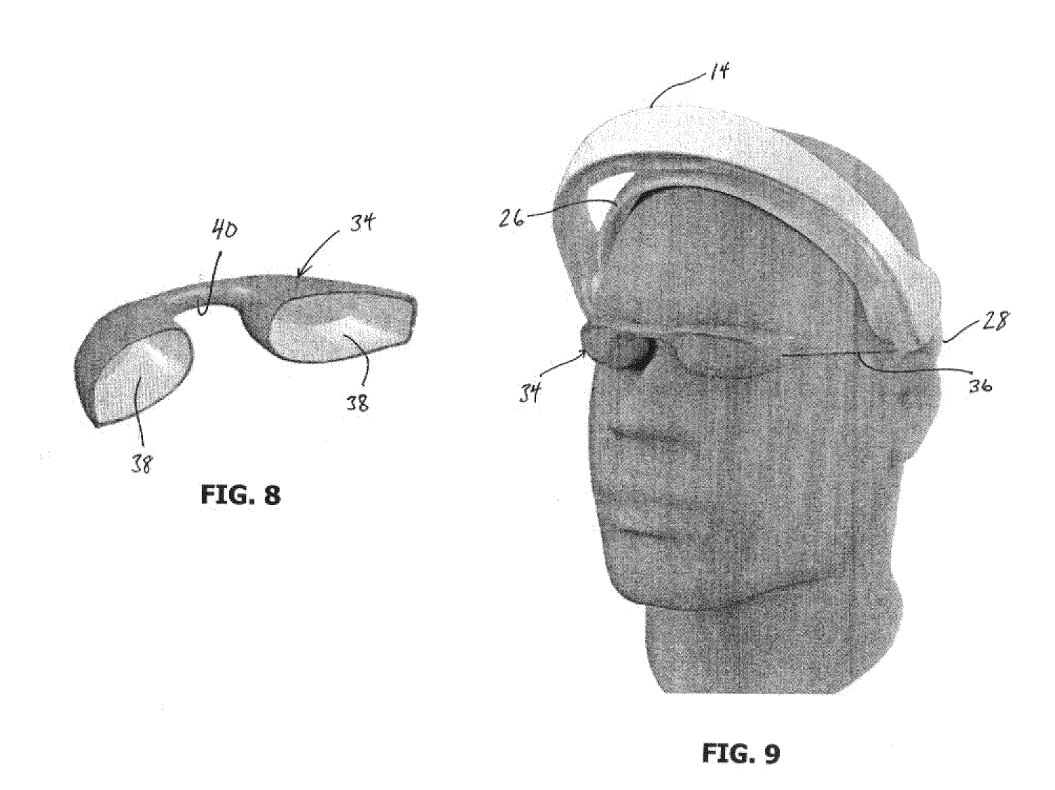

Referring to FIGS. 8 and 9, a set of goggles 34 may be used for protecting the user's eyes from UV light. A stretch cord 36 made from an elastic material (e.g., nylon) and secured to opposite ends of the goggles 34 is sized to wrap around the user's head for securing the goggles 34 against the user's face around the eyes. The inner facing walls 38 defining each of the inner cavities of the eye-covering segments of the goggles 34 may be metal-coated for added protection from UV light. In a preferred embodiment of the phototherapy apparatus 10, the light emitting diodes 102 on plate 14 are operational only if the goggles 34 are detected on the user's face in order to ensure that the user wears the goggles 34 during operation of the phototherapy apparatus 10. A triggering mechanism includes a first component on the goggles 34 that is detectable by a second component on the light emitting plate 14 when the goggles 34 are on the user's face. The light generating sources 102 are operational only when the first component is detected by the second component. In one embodiment, the first component is a magnetic strip 40 on the goggles 34 that triggers a switch (i.e., second component) on the plate 14 for turning on the light emitting diodes 102 when the user is wearing the goggles 34. Other trigger mechanisms (e.g., Bluetooth) for linking the goggles 34 and light emitting diodes 102 may be used as well.

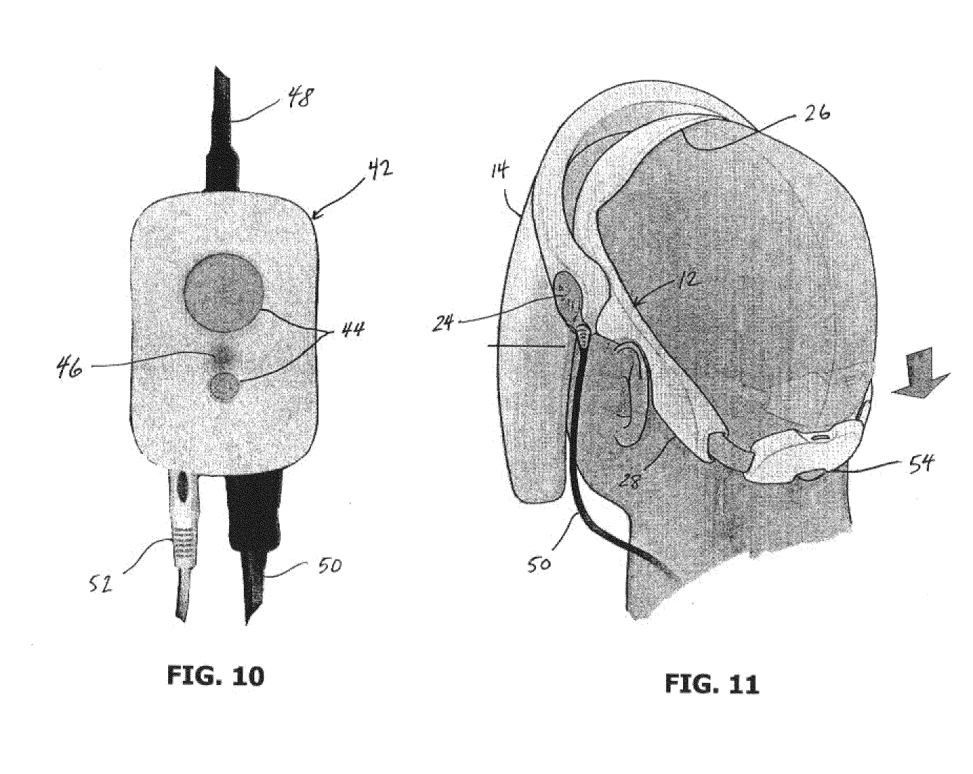

Referring to FIG. 10, a handheld controller 42 includes one or more operating controls 44 (e.g., on/off button) for operating the phototherapy apparatus 10. The controller 42 may further include one or more indicator lights 46 for signifying a function and/or error reading of the phototherapy apparatus 10. A power cord 48 extends from the controller 42 and is in connection with a plug-in power adaptor for powering the phototherapy apparatus 10. A controller cord 50 extends from the controller 42 and is in connection with the plate 14 for delivering operating signals to the light generating sources 102. An audio cord 52 is shown extending from the controller 42 and is in connection with a music playing device. While the controller 42 shown in FIG. 11 includes cords 50 and 52 for a wired connection between the controller 42 and phototherapy apparatus 10 and music playing device, respectively, the controller 42 may be structured for wirelessly operating the phototherapy apparatus 10. In another embodiment of the phototherapy apparatus 10, the operating controls 44 are located on the light emitting plate 14.

Referring to FIG. 11, one embodiment of the adjustable strap 28 is shown, wherein a dial knob 54 is provided for adjusting the fit of the supporting member 12 around the user's head. Rotation of the dial knob 54 in a first direction causes a portion of the adjustable strap 28 to wind up for tightening the adjustable strap 28 and rotation of the dial knob in an opposite direction causes a portion of the adjustable strap 28 to unwind for loosening the adjustable strap 28. Further embodiments of the adjustable strap 28 have been considered as well, such as, but not limited to, an adjustable strap 28 that is at least partially elastomeric and an adjustable strap 28 that includes two separate strap portions each including corresponding segments of a hook and loop fastener.

While the composition of the present invention has been described and exemplified according to several preferred embodiments thereof, it is recognized that departures from the instant disclosure are fully contemplated within the spirit and scope of the invention which is not to be limited except as defined in the following claims as interpreted under the Doctrine of Equivalents.

What is claimed is:

1. A wearable hands-free apparatus for providing phototherapy treatment to a user, said apparatus comprising:
a light emitting plate including an inner facing side and an outer facing side, and said light emitting plate being adapted for being supported on the user's head so that the inner facing side of said light emitting plate is positionable in spaced, opposing relation to the user's face;
an array of light generating sources on the inner facing side of said light emitting plate and said array of light generating sources being positioned, structured and disposed for producing a light pattern that can be directed onto the user's skin surface, and each of said light generating sources being further structured and disposed for emitting light within a wavelength range according to a particular condition being treated by phototherapy using the apparatus;
a set of goggles that is sized and configured for being worn on the head of the user, and said set of goggles including a first eye-covering segment and a second eye-covering segment that are each positionable over a corresponding eye of the user, and each of the first and second eye-covering segments having an inner facing wall defining an inner cavity; and
at least one control for controlling operation of each of the light generating sources in said array of light generating sources.

2. The apparatus as recited in claim 1 further comprising a triggering mechanism, and said trigger mechansim comprising a first component on said set of goggles and a second component on said light emitting plate, and said first component being detectable by said second component when said set of goggles is worn by the user, and wherein each of the light generating sources in said array of light generating sources is operational only when said first component is detected by said second component.

3. The apparatus as recited in claim 2 wherein said first component on said goggles is a magnetic strip and said second component on said light emitting plate is a switch that is structured and disposed for sending a signal to each of said light generating sources in said array of light generating sources when said magnetic strip is detected.

4. The apparatus as recited in claim 1 wherein said light emitting plate further comprises at least one vent opening extending between the inner and outer facing sides of said light emitting plate, and said at least one vent opening being sized to allow for circulation of ambient air between the inner and outer facing sides of said light emitting plate.

5. A wearable hands-free apparatus for providing phototherapy treatment to a user, said apparatus comprising:
a light emitting plate including an inner facing side and an outer facing side;
a supporting member adapted for pivotally supporting said light emitting plate on the user's head so that the inner facing side of said light emitting plate is positionable in spaced, opposing relation to a skin surface on the user's head;
an array of light generating sources on the inner facing side of said light emitting plate and said array of light generating sources being positioned, structured and disposed for producing a light pattern that can be directed onto the user's skin surface, and each of said light generating sources being further structured and disposed for emitting light within a wavelength range according to a particular condition being treated by phototherapy using the apparatus;
a set of goggles that is sized and configured for being worn on the head of the user, and said set of goggles including a first eye-covering segment and a second eye-covering segment that are each positionable over a corresponding eye of the user, and each of the first and second eye-covering segments having an inner facing wall defining an inner cavity;
at least one control for controlling operation of each of the light generating sources in said array of light generating sources; and
a triggering mechanism comprising a first component on said set of goggles and a second component on said light emitting plate, and said first component being detectable by said second component when said set of goggles is worn by the user, and wherein each of the light generating sources in said array of light generating sources is operational only when said first component is detected by said second component.

6. The apparatus as recited in claim 5 wherein said first component on said goggles is a magnetic strip and said second component on said light emitting plate is a switch that is structured and disposed for sending a signal to each of said light generating sources in said array of light generating sources when said magnetic strip is detected.

7. The apparatus as recited in claim 5 wherein said light emitting plate further comprises at least one vent opening extending between the inner and outer facing sides of said light emitting plate, and said at least one vent opening being sized to allow for circulation of ambient air between the inner and outer facing sides of said light emitting plate.

8. The apparatus as recited in claim 7 wherein said at least one vent opening is at a location on said light emitting plate that corresponds with the location of the user's mouth when said light emitting plate is positioned in spaced, opposing relation to the user's skin.

9. The apparatus as recited in claim 7 wherein said at least one vent is located on the peripheral edge of said light emitting plate.

10. The apparatus as recited in claim 5 wherein each of the inner facing walls of the first and second eye-covering segments are coated with a UV light resistant material.

11. The apparatus as recited in claim 5 wherein said supporting member comprises a first detachable strap and a second detachable strap each being formed from a semi-rigid material that is shaped to conform to the contour of a corresponding one of the user's shoulders, and said first and second detachable straps each including respective segments of a fastening member for fastening said first detachable strap to said second detachable strap and thereby securing said supporting member to the user.

12. The apparatus as recited in claim 5 wherein said supporting member comprises:
a head cup member having an inner facing side that is sized and configured for engaged receipt of the user's forehead; and
an adjustable strap having opposing ends that are integrally attached to said head cup member, and said adjustable strap being sized and configured for wrapping around the backside of the user's head for securing said supporting member to the user's head.

13. The apparatus as recited in claim 12 wherein said adjustable strap is an elastomeric strap that is structured and disposed for wrapping around and conforming to the backside of the user's head.

14. The apparatus as recited in claim 12 wherein said adjustable strap further comprises a dial knob that is structured and disposed for selectively tightening and loosening said adjustable strap, and wherein rotating said dial knob in a first direction causes a portion of said adjustable strap to wind up for tightening said adjustable strap and rotating said dial knob in an opposite direction causes the portion of said adjustable strap to unwind for loosening said adjustable strap.

15. The apparatus as recited in claim 5 wherein said light generating sources are light emitting diodes (LEDs).

16. The apparatus as recited in claim 5 wherein said light generating sources are laser diodes.

17. The apparatus as recited in claim 5 wherein said light generating sources are intense pulse lights (IPLs).

18. The apparatus as recited in claim 5 wherein said light generating sources are infrared lights.

19. The apparatus as recited in claim 5 wherein said at least one control is on a handheld controller.

20. The apparatus as recited in claim 5 wherein said at least one control is on said light emitting plate.
